(19) United States
(12) Patent Application Publication
(10) Pub. No.: US 2025/0041110 A1
(43) Pub. Date: Feb. 6, 2025

(54) MEMBRANE ACTUATED HYDRODYNAMIC DISPENSING SYSTEM

(71) Applicant: Bausch + Lomb Ireland Limited, Dublin (IE)

(72) Inventors: Yehuda IVRI, Newport Coast, CA (US); Reynaldo QUINTANA, Menlo Park, CA (US)

(21) Appl. No.: 18/848,381

(22) PCT Filed: Mar. 24, 2023
(86) PCT No.: PCT/US23/64927

§ 371 Date: Sep. 18, 2024

Related U.S. Application Data
(60) Provisional application No. 63/323,859, filed on Mar. 25, 2022.

(51) Int. Cl. A61F 9/00; A61M 11/00

(52) U.S. Cl. CPC A61F 9/0008; A61M 11/005; A61M 2205/123; A61M 2205/586

(57) ABSTRACT

Membrane actuated hydrodynamic dispensing system devices are disclosed. Aspects of the dispensing devices include an ampoule assembly and an actuator. The ampoule assembly contains liquid to be dispensed and includes the actuated membrane, one or more flexible apertures, and a stationary member to seal the aperture. The actuator is configured to vibrate the membrane to hydrodynamic excite the fluid contained in the ampoule assembly. Once the fluid is hydrodynamically excited sufficiently, the liquid is dispensed out of the aperture.

### CROSS-REFERENCE TO RELATED APPLICATION(S)

This application claims priority to, and the benefit of, U.S. Provisional Application No. 63/323,859, filed Mar. 25, 2022, for all subject matter common to both applications. The disclosure of said provisional application is hereby incorporated by reference in its entirety.

### FIELD OF THE INVENTION

The present invention generally pertains to devices for dispensing fluid medicines and more particularly pertains to such devices that store and deliver ophthalmic preservative-free medicines, specifically configured to increase the ease of use, and enhance patient compliance with dosing instructions for the medicine.

### BACKGROUND

There are a number of prescription drugs available for topical application to the eye to treat a variety of ailments, diseases, and conditions. The standard applicator for ophthalmic solutions is the conventional eyedropper. When using an eyedropper, the drug is taken routinely from the same bottle with no control as to the ingress of outside particulate from entering the bottle. When a conventional eyedropper is used, it can require a high level of dexterity and control to properly apply the drop accurately to the eye. Compounding this issue is that if the eye requires medication, it is not operating at normal effectiveness which further decreases the accuracy of the user. This results in ophthalmic medication being misapplied, not applying the intended amount of medication. For example, a user may apply too much medication, i.e., too many drops, or not enough medication due to misapplication.

Ease of dispensing fluid medicines and compliance with dosing instructions are primary concerns with all patients. For example, dispensing bottles such as ophthalmic squeeze dispensers typically require greater actuation force due to a valve mechanism that seals the dispensing nozzle to prevent bacterial ingress and contamination. Such a system requires much higher pressure to operate, and hence much higher squeeze force is required. In addition, prior art dispensing bottles dispense only in an upside-down orientation which require an inconvenient head maneuver and which together with higher actuation force further increases the inconvenience.

Further, the drug is applied through the force of gravity, carrying the drop or droplets into the eye. Then, after the drop is delivered, a portion of the drug is taken back into the container due to the negative pressure within the eyedropper when the eye dropper is released. The portion of drug “sucked” back into the container can introduce microbials and unwanted dust or particulate into the container.

Another concern is shelf-life of ophthalmic solutions. To increase shelf-life of these ophthalmic solutions and to combat any infectious agents, preservatives and other agents are introduced into the ophthalmic solutions. These additives can have various negative side effects experienced by the patient or end-user of the ophthalmic medication. For example, some of the most well used preservatives can cause irritation, dry eye symptoms, effects to tear production and maintenance of the tear film, among others.

As such, there is a current, and pressing, need for a solution that can accurately deliver ophthalmic solution while allowing a preservative free solution to be used.

### SUMMARY

In some aspects, the techniques described herein relate to a handheld device for dispensing a liquid to an eye of a patient, the device including: an ampoule containing the liquid to be dispensed; an assembly connected to the ampoule and in fluid communication therewith, the assembly having (a) one or more flexible apertures through which fluid from the ampoule can be dispensed, (b) a member for sealing engagement against the one or more flexible apertures, and (c) a membrane, which when acted upon, creates pressure toward the one or more flexible apertures to disengage the one or more flexible apertures from the member to dispense the liquid from the ampoule therethrough; and an actuator removably coupled to the assembly and designed to impart vibrations to the membrane so that sufficient pressure can be generated to disengage the one or more flexible apertures from the member and to push the fluid through the one or more flexible apertures to dispense it from the assembly.

In some aspects, the techniques described herein relate to a device, wherein the one or more flexible apertures defines a fluid exit pathway that decreases in diameter from its proximal end to its distal end.

In some aspects, the techniques described herein relate to a device, wherein the decrease in diameter of the fluid exit pathway imparts an increase in fluid velocity as the fluid travels along the exit pathway and out through the one more flexible apertures.

In some aspects, the techniques described herein relate to a device, wherein the member is stationary, relative to the assembly, to maintain a force against the one or more flexible apertures while in sealed engagement.

In some aspects, the techniques described herein relate to a device, wherein the member has a tip designed to be in engagement with the one or more flexible apertures in a fluid tight manner.

In some aspects, the techniques described herein relate to a device, wherein the membrane can be acted upon by the actuator to have an oscillation frequency of greater than 80 cycles per second and an amplitude is less than 0.2 mm.

In some aspects, the techniques described herein relate to a device, the device further including: a cover positioned over the one or more flexible apertures when the device is not in use to prevent particulate and residue from gathering on the one or more flexible apertures, where the cover can be adjusted to an open position when the device is in use.

In some aspects, the techniques described herein relate to a device, the cover further including: a plug disposed on the cover designed to be inserted into the one or more flexible apertures to seal and clear the one or more flexible apertures.

In some aspects, the techniques described herein relate to a device, wherein the coupling of the actuator to the assembly includes an interference fit.

In some aspects, the techniques described herein relate to a device, the device further including: a first cylindrical interfacing member disposed proximate to the membrane; and a second cylindrical interfacing member disposed proximate to the actuator, the second cylindrical interfacing member designed to couple with the first cylindrical interfacing member to mechanically couple the assembly and the actuator.

In some aspects, the techniques described herein relate to a device, the device further including: an ergonomically designed housing surrounding the device, the housing having an opening to permit the liquid to be dispensed from the one or more flexible apertures while the device is contained within the housing, the housing includes two parts coupled together such that the assembly is removable from within the housing.

In some aspects, the techniques described herein relate to a system for dispensing a liquid to a target location, the system including: a first assembly having (a) an ampoule containing the liquid to be dispensed, (b) an aperture through which liquid from the ampoule can be dispensed to the target location, (c) a stationary member disposed within the ampoule for sealing the aperture, and (d) a membrane designed to excite the liquid in the ampoule to dispense the liquid through the aperture; and a second assembly having an actuator which imparts vibrations to the membrane to excite and dispense the liquid through the aperture, the second assembly is mechanically coupled to the first assembly.

In some aspects, the techniques described herein relate to a system, wherein the stationary member has a tip that is in fluid tight engagement with the aperture when the actuator is not vibrating.

In some aspects, the techniques described herein relate to a system, wherein the aperture defines a fluid exit pathway that decreases in diameter from its proximal end to its distal end.

In some aspects, the techniques described herein relate to a system, wherein the decrease in diameter of the fluid exit pathway imparts an increase in fluid velocity as the fluid travels along the exit pathway and out through the aperture.

In some aspects, the techniques described herein relate to a system, the system further including: an ergonomically designed housing surrounding the system, the housing having an opening to permit the liquid to be dispensed from the aperture, the housing includes two parts coupled together such that the first assembly is removable from within the housing.

In some aspects, the techniques described herein relate to a system, the system further including: a cover positioned over the aperture when the system is not in use to prevent particulate and residue from gathering on the aperture, where the cover is adjustable to an open position when the system is in use.

In some aspects, the techniques described herein relate to a system, the cover further including: a plug disposed on the cover designed to be inserted into the aperture to seal and clear the aperture.

In some aspects, the techniques described herein relate to a system, wherein the coupling of the first assembly to the second assembly includes an interference fit.

In some aspects, the techniques described herein relate to a system, wherein the membrane can be acted upon by the actuator to have an oscillation frequency of greater than 80 cycles per second and an amplitude is less than 0.2 mm.

### BRIEF DESCRIPTION OF DRAWINGS

These and other characteristics of the present disclosure will be more fully understood by reference to the following detailed description in conjunction with the attached drawings, in which:

FIG. 1 is a cross sectional view of an embodiment of the invention.

FIG. 2 is a cross sectional view of the embodiment showing the valve in an open state.

FIG. 3 is a cross sectional view of the dispensing embodiment with a removable pivoted actuator of FIG. 1 with removable oscillator.

FIG. 4A is a cross sectional view of a first assembly of the removable actuator of FIG. 3.

FIG. 4B is a cross sectional view of a second assembly of the removable ampoule of FIG. 3.

FIG. 5 is side view of an embodiment contained in a housing.

FIG. 6 is a side view of an embodiment contained in a housing illustrating a removable ampoule.

FIG. 7 is an illustration of dosage consistency per shot across various devices.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure relates to handheld devices for dispensing liquids to a desired location, e.g., an eye of a patient. For example, the present disclosure relates to a device with an ampoule for containing liquid, an assembly with a membrane, which when acted upon, can create hydrodynamic pressure towards an aperture being sealed by a member. An actuator can impart vibrations to the membrane to create the hydrodynamic excitation in the fluid. When the excitation reaches a predetermined threshold value, the aperture can be deformed such that it can be separated from the member or pin and liquid can be dispensed therethrough.

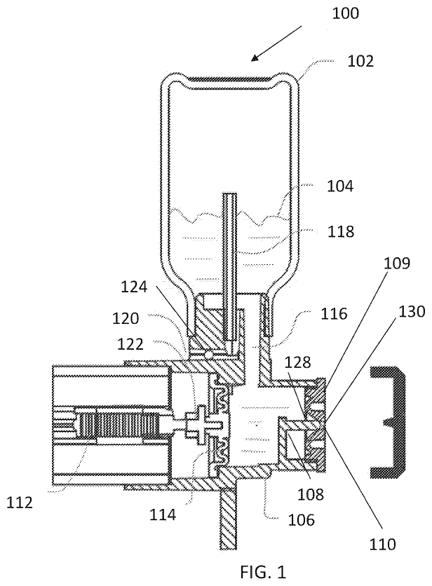

FIGS. 1-6, wherein like parts are designated by like reference numerals throughout, illustrate an example embodiment or embodiments of the device of dispensing liquid to an eye, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

Referring to FIG. 1, in some embodiments, the present disclosure provides a handheld device 100 for dispensing liquid 104, e.g., an ophthalmic medication or other therapeutic, to an eye of a patient or a site of interest. Generally, the liquid dispensing device 100 can include an ampoule 102 for containing the liquid 104 to be dispensed to the ocular surface, or a site of interest. The handheld device 100 can include a chamber 106 which can be connected to, and can be in fluid communication with, the ampoule 102 by a channel or fluid pathway 116. In an embodiment, liquid 104 can be drawn, e.g., by gravity, through the fluid pathway 116 into the chamber 106 for dispensing. In an embodiment, the dispensed liquid 104 has a volume of about 5 μl to about 30 μl. In some embodiments, the dispensed liquid can have a volume of about 12 μl.

To prevent creation of a pressure vacuum within the ampoule 102, as fluid flows from ampoule 102 to chamber 106, in some embodiments, a vent 118 can be included in the device 100 which can equalize the pressure in the ampoule 102 through the introduction of air from the vent 118. The vent 118 can be connected to atmospheric pressure at vent outlet 120. To prevent the introduction of most, if not all, contaminants or bacteria, a filter 124 can be included in vent outlet 120. The filter 124 can include any or a combination of filters known in the art for filtering microbes, or other unwanted particulates, from air.

Alternatively, or additionally, to prevent ingress of contaminants into the handheld device 100, the device 100 can include a sealed engagement between an aperture plate, or plate 109, and a member, or pin 108. The sealed engagement of the aperture plate and the pin 108 can provide a selectively closed seal at an outlet, or aperture 110 to the chamber 106. The sealed engagement of chamber 106 can be akin to a check valve. As seen in FIG. 1, the plate 109 can include the aperture 110 for dispensing liquid 104 therethrough. The sealed engagement between the pin 108 and the aperture 110 can create a hermetic fluid tight seal. However, the sealed engagement between the pin 108 and aperture 110 can be opened, or disengaged, when a hydrodynamic excitation of the liquid 104 in the chamber 106 reaches a threshold value to open the aperture 110. When the pin 108 and aperture 110 are disengaged, the liquid 104 can be dispensed through the aperture 110. If the pressure is below the threshold value, the liquid 104 in chamber 106 can be sealed within the chamber 106, or travel through the pathway 116 up to the ampoule 102. In some embodiments, the aperture 110 can be disengaged from the pin 108 when the pressure within the chamber 106 exceeds about 0.014 MPa to about 0.04 MPa. In some embodiments, the plate 109 can be made of a flexible elastomer for the sealed engagement. In an embodiment the elastomer can be silicone rubber. Other elastomers which can have a Youngs modulus of elasticity of about 0.1 to about 1.2 GPa can also be used. In some embodiments, the plate 109 can include more than one aperture 110. In other embodiments, the seal between pin 108 and aperture 110 can be any seal.

As can be seen in FIG. 1, in some embodiments, the aperture 110 can be conical and can extend through the plate 109. In other embodiments, the aperture 110 can extend through a side wall of the chamber 106. The conical aperture 110 can have a large inlet opening 128 in fluid communication with the chamber 106 and small exit opening 130 through which the liquid 104 can be dispensed. The conical shape can allow the pin 108 to engage with the aperture 110 and allow a more controlled dispensing of liquid 104 through the aperture 110 as the aperture 110 is disengaged from the pin 108. The conical shape of the aperture 110 can have a diameter of about 0.1 mm to about 0.6 mm at a small end, or exit opening 130, and about 0.7 mm to about 1.0 mm at a large end, or inlet opening 128. In an embodiment, the conical shape of the aperture 110 can have a diameter of about 0.2 mm at the exit opening 130 and a diameter of about 0.8 mm at the inlet opening 128. Alternatively, in some embodiments, the aperture can have a tubular, tapered, or other shape. The decreasing diameter of the fluid exit pathway in the aperture 110 can impart an increase in fluid velocity as the fluid travels along the exit pathway and out through the aperture 110.

Still in reference to FIG. 1, the pin 108 that is in sealed engagement with the aperture 110 can be stationary, relative to the chamber 106, to maintain a force against the aperture 110 while in sealed engagement. The aperture 110 can disengage from the pin 108, when the hydrodynamic pressure within the chamber 106 exceeds a threshold value, upon actuation of the device 100. In the illustrated embodiment, the stationary pin 108 can have an ‘L’ shape and can be affixed to the bottom of the chamber 106 to create the sealed engagement with aperture 110. Alternatively, in other embodiments, the stationary pin 108 can be straight and be disposed opposite aperture 110. In still other embodiments, the pin 108 can be disposed anywhere capable of maintaining the sealed engagement with aperture 110.

In some embodiments, the pin 108 can have a spherical tip designed to be received by the aperture 110. The spherical tip on pin 108 can create a sealing engagement with the aperture 110 that can be more controllably disengaged to dispense liquid 104 therethrough. In some embodiments, the spherical tip of pin 108 and the conical aperture 110 can engage in a complimentary fashion. In alternative embodiments, any complementary geometric tip, can be contemplated in place of the spherical tip on pin 108, for example a conical tip or tubular tip. In still other embodiments, non-complementary surfaces or tips can be used. In some embodiments, the spherical member may include an antibacterial coating or made of an antibacterial material.

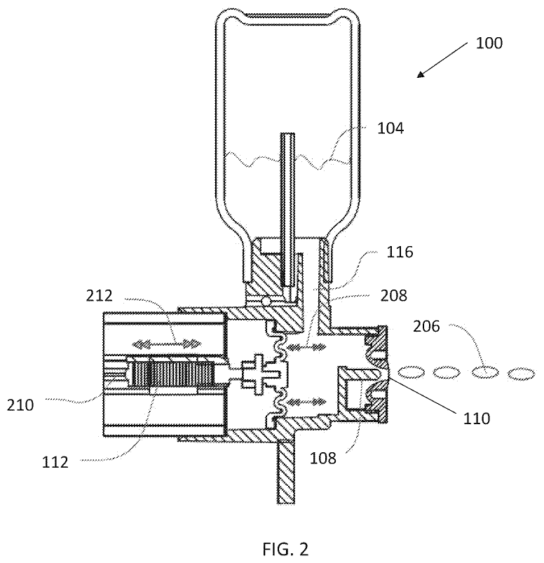

In some embodiments, to hydrodynamically excited the liquid 104 in the chamber 106, the device 100 can include a membrane 114 which, when acted upon or actuated, can create fluid momentum within the liquid 104. The fluid momentum in the liquid 104 can increase the hydrodynamic pressure of the fluid within chamber 106. To generate fluid momentum, the membrane 114 can be oscillated, vibrated, or otherwise acted upon by an actuator 112. In reference to FIG. 2, the vibrations 212 or oscillations imparted to the membrane 114 can create cycles of hydrodynamic pulses to create momentum in the liquid 104, causing the aperture 110 to disengage from the pin 108 as the fluid pushes against the aperture 110. For example, in some embodiments the pulse displacement of membrane 114 is about 100 microns to about 200 microns and the pulse is about 100 ms. The aperture 110 can be re-sealed by the pin 108 when the fluid pressure is reduced. Alternatively, in some embodiments, the membrane 114 can be a rigid wall, semi-rigid wall, the wall of the actuator 112, or any wall that can be acted upon, or vibrated, to hydrodynamically excite the liquid 104.

Still in reference to FIG. 2, a cross-sectional view of the handheld device 100 in a dispensing configuration can be observed. In some embodiments, the device 100 can include an actuator 112 removably coupled to the chamber 106 and the actuator 112 can be designed to impart vibrations 212 to the membrane 114. The vibrations 212 to the membrane 114 can increase the pressure in chamber 106 to disengage the aperture 110 from the pin 108, dispensing liquid 104 therethrough. The actuator 112 can be connected to membrane 114 through a connecting member, or connecting rod 122. As the actuator 112 oscillates along actuator track 210, the connecting rod 122 can impart the vibrations 212 to the membrane 114 to hydrodynamically excite the liquid 104 in the chamber 106 as shown by arrows 208. In some embodiments, this actuation can dispense the liquid 104 as droplets 206 or as a continuous stream of liquid 104. In an embodiment, the connecting rod 122 can be designed to modulate the vibrations from the actuator 112 to deliver a steadier application of force from actuator 112.

In some embodiments, the oscillation frequency of the actuator 112, which can be a vibrational motor, or coin-cell motor, can actuate the membrane 114 to about 80 revolutions per second, or greater, with an amplitude between about 0.2 mm to about 0.5 mm to generate the fluid momentum necessary to open the aperture 110. Alternatively, the amplitude may be less than about 0.2 mm. In some embodiments, if the membrane 114 is actuated less than about 50 cycles per second, the liquid 104 can be displaced from the chamber 106 back through pathway 116 to the ampoule 102, rather than being dispensed through the aperture 110. This embodiment can prevent ingress of microorganisms into the chamber 106 by preventing inadvertent disengagement of the aperture 110 from the pin 108. Alternatively, in other embodiments, any actuator 112 or motor with a similar function can be substituted for the actuator 112. For example, in some embodiments, the actuator 112 can be a concealed motor assembly such as a coin vibrational motor, such as part number: DM-B1002-30 manufactured by Zhejiang Dongyang Dmegc Chengji Electronics Co., Ltd. China. In other embodiments, membrane vibration can also be produced by an electromagnetic or piezoelectric transducer.

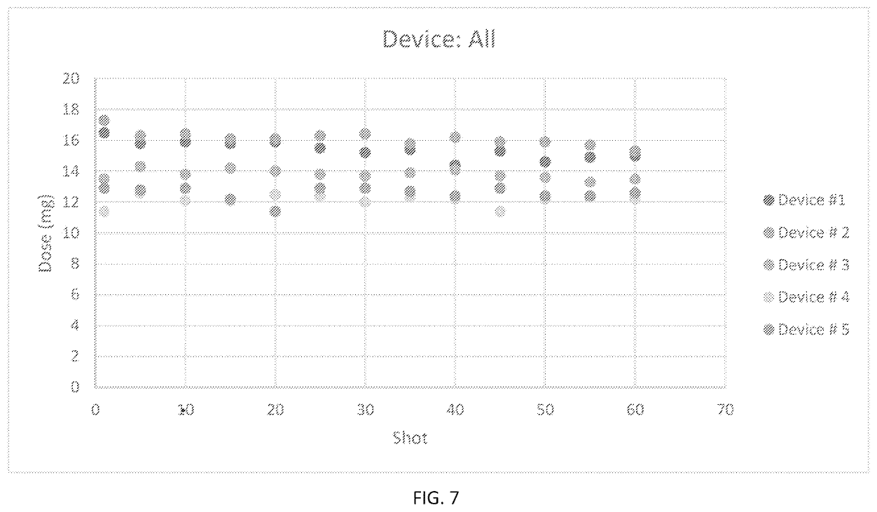

In an embodiment, the actuator 112 can be controlled by a microprocessor to produce consistent flow rate and dose output across varying configurations of the device 100. Dose consistency across various devices is illustrated in FIG. 7. For example, the microprocessor can detect the actuator 112 currently configured, through a known database, and control actuator 112 to deliver a consistent volume of liquid 104 using the database to determine how to control the actuator 112. To control the actuator 112, pulse-width-modulation (PWM) can be used. In some embodiments, the control can be through limiting voltage being supplied the actuator 112. For example, in some embodiments the input voltage can be approximately 8 volts to approximately 12 volts. The input voltage can vary depending on the viscosity of the liquid 104. In some embodiments the ON time for the actuator can be less than approximately 100 ms. Additionally, to provide feedback to the microprocessor, a sensor (not shown) can be included in the device 100. The sensor can detect the speed, or rotation, or vibrations, of the actuator 112 and provide this measurement to the microprocessor. The measurement can be used by the microprocessor to either limit or increase the actuation speed of actuator 112. In some embodiments, the microprocessor can be microprocessor PIC16F1509T-/SO manufactured by Microchip Technology Chandler, Arizona USA.

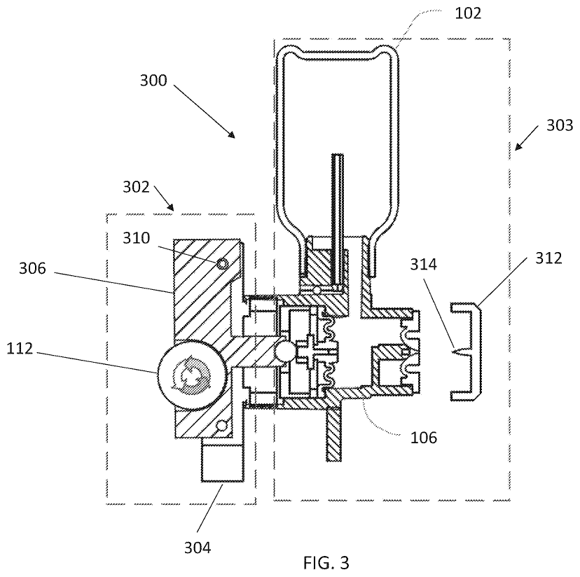

Referring now to FIG. 3, an alternative embodiment is shown illustrating a device 300 that can be separated into a first assembly, or an actuator assembly 302, from a second assembly, or an ampoule assembly 303. This modular design can have a number of benefits. For example, another vector for microorganisms to enter the liquid 104 in device 100 can be through refilling the ampoule 102 by the patient or user. To prevent this microorganism ingress, in some embodiments, it can be beneficial to provide a disposal ampoule assembly 303 to replace an ampoule 102 that has run out of liquid 104. As such, it can be appreciated that the separability of the ampoule assembly 303 and actuator assembly 302 can result in health benefits by preventing negative outcomes from having a user refill ampoule 102 while preventing unnecessary waste by having to replace the entire device 300. In some embodiments, alternatively, the entire device 300 can be disposable. However, separation between actuator assembly 302 and ampoule assembly 303 can reduce waste by reusing the actuator assembly 302 with a replaceable ampoule assembly 303. Reusing the actuator assembly 302 can prevent waste and decrease cost of the device 300. Replacing the ampoule assembly 303 eliminates a vector of microorganism ingress into the device 300. While in some embodiments, the ampoule assembly 303 can be disposable, in other embodiments, the actuator assembly 302 can be disposable. Further, the handheld dispensing system, or device 300, can be designed to be permit ease of separation between ampoule assembly 303 and actuator assembly 302. For example, in some embodiments, the actuator assembly 302 can be connected to the ampoule assembly 303 by an interference fit. In other embodiments, the actuator assembly 302 can be fastened or affixed to the ampoule assembly 303 using other mechanical fasteners, e.g., magnets, springs, pivotable pins, etc., that can still permit ease of removal.

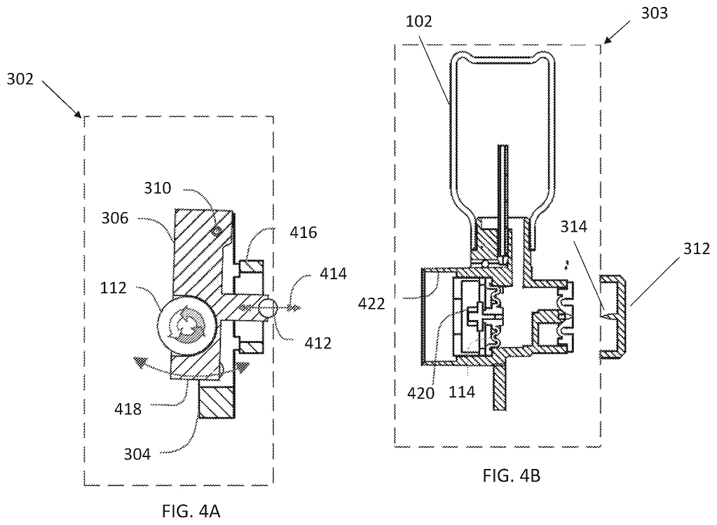

Referring now to FIG. 4A and FIG. 4B, in some embodiments, the device 300 can include a first cylindrical interfacing member 422 disposed on the ampoule assembly 303, proximate to the membrane 114, and a second cylindrical interfacing member 416 disposed on the actuator assembly 302, proximate to the actuator 112. The second cylindrical interfacing member 416 can be designed to couple with the first cylindrical interfacing member 422 to mechanically, or operably, be coupled together by engagement between the first cylindrical interfacing member 422 and the second cylindrical interfacing member 416. The cylindrical shapes of the first cylindrical interfacing member 422 and the second cylindrical interfacing member 416 can permit ease of attachment while providing sufficient force to maintain the engagement therebetween. Alternatively, in other embodiments, any shape or combination of shapes or coupling mechanisms can be used in addition to, or in place of, first cylindrical interfacing member 422 and second cylindrical interfacing member 416.

In some embodiments, device 300 can include an actuator 112 as shown in FIGS. 1 and 2, e.g., a vibratory motor. Alternatively, the actuator 112 can be a pivoting actuator 112, as illustrated in FIGS. 3, 4A, and 4B. The pivoting actuator 112 can be arranged within the actuator assembly 302 and can, in general, include a pivot pin 310 disposed in the actuator 112 through a base member 304. The pivoting actuator can additionally include a swing member 306 pivotally arranged on the pivot pin 310 to allow the swing member 306 to rotate about the pivot pin 310. As the swing member 306 rotates about pivot pin 310 the swing member 306 can vibrate the membrane 114 due to impact of the swing member 306 against the membrane 114. The rotation of swing member 306 can be in the direction illustrated by swing member movement 418. In an embodiment, the swing member angular movement 418 can be less than about a fraction of a degree.

In some embodiments, the actuator 112 of the handheld device 300 can include a first magnet 412 arranged, or disposed, on the swing member 306 and a second magnet 420 can be disposed on the membrane 114. The first and second magnets 412, 420 can be arranged such that they are co-axial, or in alignment to come into contact with one another. For example, the first magnet 412 can engage the second magnet 420 disposed on the membrane 114, such that the first magnet 412 magnetically pushes and pulls the second magnet 420 as the swing member 306 is actuated. As the swing member 306 is rotated about pivot pin 310, the distance between magnet 412 and the second magnet 420 can change to increase and decrease the magnetic force applied to the second magnet 420. In some embodiments, the first magnet 412 and second magnet 420 can further provide a dampening force from the actuator 112 to the membrane 114 to prevent excessive forces on the membrane 114. Additionally, the first magnet 412 can operably engage with the second magnet 420 when the actuator assembly 302 and an ampoule assembly 303 are connected or engaged. This can reduce the tolerances required for the coupling mechanism between actuator assembly 302 and ampoule assembly 303 as the magnetic force between first magnet 412 and second magnet 420 will maintain connection between the actuator assembly 302 and ampoule assembly 303. In an embodiment, the amplitude of magnetic movement 414 can be less than about 0.5 mm. Alternatively, in other embodiments, any mechanism or adhesives that can allow for attaching and detaching of actuator assembly 302 and ampoule assembly 303 can be substituted.

Referring to FIGS. 3 and 4B, in some embodiments, the device 300 can include a cover 312, or cap, affixed to an exterior of the chamber 106 to cover the aperture 110 when the device 300 is not in use. The cover 312 can be removed to use the device 300. The cover 312 can prevent dust, particulate, sediment, or any other unwanted residue from settling on or potentially entering the aperture 110 while the device is not in use. To prevent unwanted ingress of particulates or microbes, the cover 312 can, in some embodiments, be a screw cap or lid that can be removably affixed to shield the aperture 110. In alternative embodiments, the cover 312 can function as an ergonomic guide to position the device 300, for example, as an initial alignment method to align the aperture 110 with the target to receive the dispensed liquid 104. While illustrated as a separatable cover 312, it should be understood that, in alternative embodiments, the cover 312 can be hingedly affixed to an exterior of the device 300 or any other structural method for achieving the same functional ends as described above.

In some embodiments, the cover 312 can include a plug 314 extending from an inside surface of the cover 312. The plug 314 can be designed to be inserted into the aperture 110 to additionally seal and clear the aperture 110. To clear the aperture 110, the plug 314 can push or displace liquid 104 from the aperture 110 to prevent a build-up of fluid residue within the aperture 110. This can be beneficial when the liquid 104 may be viscous and can leave solid, or dry, residue in or on the aperture 110. Additionally, the plug 314 can prevent clogging by removing debris and particulate from aperture 110. To prevent clogging, the plug 314 can enter the aperture 110 when the cover 312 can be in the closed configuration. This can also augment the sealing engagement between the aperture 110 and pin 108, which can relieve mechanical stress on the pin 108 and the aperture 110. In alternative embodiments, cover 312 can be a sliding door, a flap that can be attached at one end, or any other structural element that can shield the aperture 110 as described above.

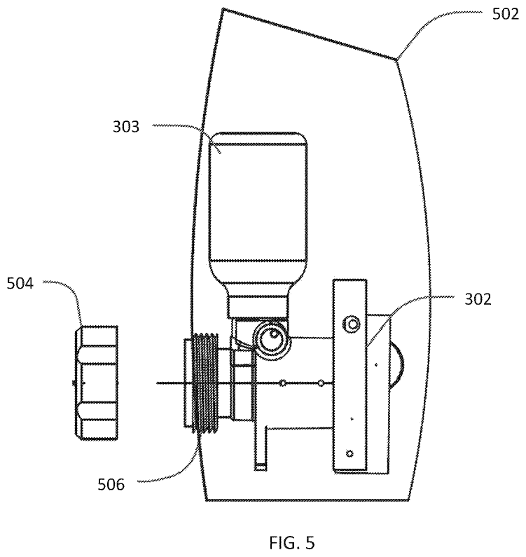

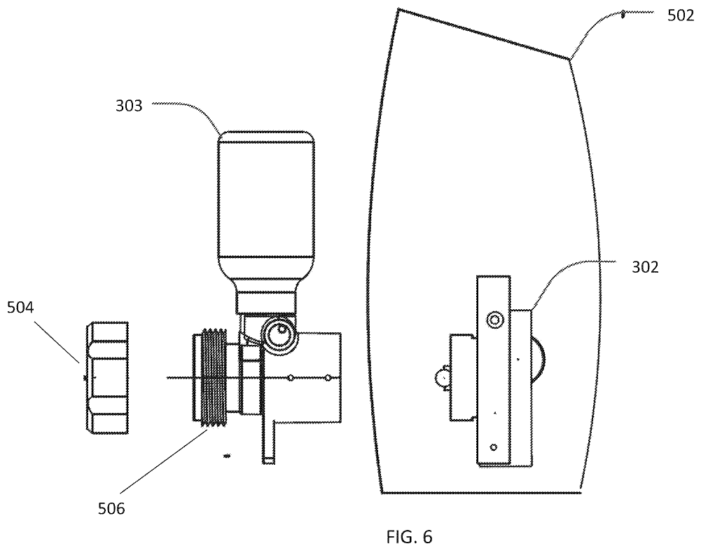

Referring now to FIGS. 5 and 6, in some embodiments, the device 100, or device 300, can include an ergonomically designed housing 502 to surround the device 100. The housing 502 can have an opening (not shown) through which the liquid 104 can be dispensed from the aperture 110. In some embodiments, the housing 502, can include two parts coupled together such that the ampoule assembly 303 can be removable from the housing 502 while the actuator assembly 302 remains affixed to an interior of the housing 502. In other embodiments, the housing 502 can be more than two parts. In alternative embodiments, the housing 502 may not completely surround the device 100 and can only cover specific portions of the device 100, such as only covering the actuator assembly 302, for example. Generally, the housing 502 can provide an ergonomic surrounding of the internal components of the device 100, such as the actuator assembly 302 and the ampoule assembly 303. For example, the housing 502 can, in some embodiments, be manufactured to be ergonomically designed to be held by a hand. For example, the shape of the housing 502 can be manufactured to improve the accuracy of the delivery of the liquid 104 to the site of interest. The accuracy can be improved, merely by way of example, by integrating the cover 312 in a way that can guide the user in a proper, or recommended, use of the device 100. To illustrate, the cover 312 can be a flap which can be used as an alignment technique to accurately deliver the liquid 104 to the eye. In an embodiment, the housing 502 can be designed to accommodate a rotatable cap, or cover 504, and threading 506. The rotatable cap or cover 504 can be attached to the housing 502 to cover the aperture 110 to prevent debris or other particulate from entering or blocking the aperture 110 and can be removed to dispense liquid 104 through the aperture 110.

Referring to FIG. 6, an embodiment of the housing 502 is shown with the ampoule assembly 303 removed from the housing 502. In some embodiments, the housing 502 can have a wall that can be removable which can allow access to the ampoule assembly 303. A removeable wall can allow a user to replace the ampoule assembly 303 when desired rather than refilling the ampoule 102 with liquid 104. Having the ampoule assembly 303 being replaceable can prevent ingress of bacteria or contaminant into the system, as discussed above. The housing 502 can have an opening (not shown) or housing 502 can be separated into parts whereby the ampoule assembly 303 can be removed from housing 502. In some embodiments, housing 502 can include an electronic circuit, printed circuit board, and a battery (not shown).

A use of an embodiment of the handled dispenser device 100 will now be described. A user can remove the cover 312 or cap from in front of the aperture 110. In other embodiments, the cover 312 remains partially affixed to the device 100. For example, the user may utilize the cover 312 to orient or align the device 100. A button (not shown) can be engaged to activate the actuator 112. The actuator 112 can begin oscillating the membrane 114, thereby hydrodynamically exciting the liquid 104 and imparting momentum to the liquid 104 contained in the chamber 106. Once the pressure in the chamber 106 exceeds a threshold, the aperture 110 will be disengaged from the pin 108, dispensing a predetermined volume of the liquid 104 contained in the chamber 106. As the pressure in the chamber 106 subsides or decreases, the aperture 110 will reengage with the pin 108 to create a hermetic fluid tight seal, preventing further liquid 104 from being dispensed. The user can optionally replace the cover 312 to the closed configuration or dispense an additional partial or full dose of the liquid 104.

This method can be repeated until the ampoule assembly 303 needs to be replaced. The user may want to replace the ampoule assembly 303 because an amount of liquid 104 has been dispensed or to switch dispensing solutions. To replace the ampoule assembly 303, the user can remove the ampoule assembly 303 from the housing 502 or the actuator assembly 302 and replace the ampoule assembly 303 with a new ampoule assembly 303. As can be appreciated the method herein described is an example and is not meant to limit or otherwise inhibit the disclosure or any embodiments disclosed herein.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

What is claimed is:

1. A handheld device for dispensing a liquid to an eye of a patient, the device comprising:
an ampoule containing the liquid to be dispensed;
an assembly connected to the ampoule and in fluid communication therewith, the assembly having (a) one or more flexible apertures through which fluid from the ampoule can be dispensed, (b) a member for sealing engagement against the one or more flexible apertures, and (c) a membrane, which when acted upon, creates pressure toward the one or more flexible apertures to disengage the one or more flexible apertures from the member to dispense the liquid from the ampoule therethrough; and
an actuator removably coupled to the assembly and designed to impart vibrations to the membrane so that sufficient pressure can be generated to disengage the one or more flexible apertures from the member and to push the fluid through the one or more flexible apertures to dispense it from the assembly.

2. The device of claim 1, wherein the one or more flexible apertures defines a fluid exit pathway that decreases in diameter from its proximal end to its distal end.

3. The device of claim 2, wherein the decrease in diameter of the fluid exit pathway imparts an increase in fluid velocity as the fluid travels along the exit pathway and out through the one more flexible apertures.

4. The device of claim 1, wherein the member is stationary, relative to the assembly, to maintain a force against the one or more flexible apertures while in sealed engagement.

5. The device of claim 1, wherein the member has a tip designed to be in engagement with the one or more flexible apertures in a fluid tight manner.

6. The device of claim 1, wherein the membrane can be acted upon by the actuator to have an oscillation frequency of greater than 80 cycles per second and an amplitude is less than 0.2 mm.

7. The device of claim 1, the device further comprising:
a cover positioned over the one or more flexible apertures when the device is not in use to prevent particulate and residue from gathering on the one or more flexible apertures, where the cover can be adjusted to an open position when the device is in use.

8. The device of claim 7, the cover further comprising:
a plug disposed on the cover designed to be inserted into the one or more flexible apertures to seal and clear the one or more flexible apertures.

9. The device of claim 1, wherein the coupling of the actuator to the assembly includes an interference fit.

10. The device of claim 1, the device further comprising:
a first cylindrical interfacing member disposed proximate to the membrane; and
a second cylindrical interfacing member disposed proximate to the actuator, the second cylindrical interfacing member designed to couple with the first cylindrical interfacing member to mechanically couple the assembly and the actuator.

11. The device of claim 1, the device further comprising:
an ergonomically designed housing surrounding the device, the housing having an opening to permit the liquid to be dispensed from the one or more flexible apertures while the device is contained within the housing, the housing includes two parts coupled together such that the assembly is removable from within the housing.

12. A system for dispensing a liquid to a target location, the system comprising:
a first assembly having (a) an ampoule containing the liquid to be dispensed, (b) an aperture through which liquid from the ampoule can be dispensed to the target location, (c) a stationary member disposed within the ampoule for sealing the aperture, and (d) a membrane designed to excite the liquid in the ampoule to dispense the liquid through the aperture; and
a second assembly having an actuator which imparts vibrations to the membrane to excite and dispense the liquid through the aperture, the second assembly is mechanically coupled to the first assembly.

13. The system of claim 12, wherein the stationary member has a tip that is in fluid tight engagement with the aperture when the actuator is not vibrating.

14. The system of claim 12, wherein the aperture defines a fluid exit pathway that decreases in diameter from its proximal end to its distal end.

15. The system of claim 14, wherein the decrease in diameter of the fluid exit pathway imparts an increase in fluid velocity as the fluid travels along the exit pathway and out through the aperture.

16. The system of claim 12, the system further comprising:
an ergonomically designed housing surrounding the system, the housing having an opening to permit the liquid to be dispensed from the aperture,
the housing includes two parts coupled together such that the first assembly is removable from within the housing.

17. The system of claim 12, the system further comprising:
a cover positioned over the aperture when the system is not in use to prevent particulate and residue from gathering on the aperture, where the cover is adjustable to an open position when the system is in use.

18. The system of claim 17, the cover further comprising:
a plug disposed on the cover designed to be inserted into the aperture to seal and clear the aperture.

19. The system of claim 12, wherein the coupling of the first assembly to the second assembly includes an interference fit.

20. The system of claim 12, wherein the membrane can be acted upon by the actuator to have an oscillation frequency of greater than 80 cycles per second and an amplitude is less than 0.2 mm.
